(19) United States
(12) Patent Application Publication
(10) Pub. No.: US 2005/0014137 A1
(43) Pub. Date: Jan. 20, 2005

(54) Lentivirus assay system including Vif protein activity

(75) Inventors: Wade Stanton Blair, San Marcos, CA (US); Joan Qun Cao, Carlsbad, CA (US); Amy Karen Patick, Escondido, CA (US)

Correspondence Address: AGOURON PHARMACEUTICALS, INC., 10350 NORTH TORREY PINES ROAD, LA JOLLA, CA, 92037, US

(73) Assignee: AGOURON PHARMACEUTICALS, INC.

(21) Appl. No.: 10/881,501

(22) Filed: Jun. 29, 2004

Related U.S. Application Data
(60) Provisional application No. 60/488,078, filed on Jul. 17, 2003.

(51) Int. Cl. C12Q 1/70; C12P 1/00; G01N 33/53; G01N 33/567

(52) U.S. Cl. 435/5; 435/7.1; 435/7.2; 435/41

(57) ABSTRACT

The present invention relates to a lentivirus co-culture assay system that can detect modulation of Vif protein activity and that can be formatted for high throughput screening to identify antiviral agents.

This application claims the benefit of U.S. Provisional application Ser. No. 60/488,078, filed Jul. 17, 2003, the contents of which is hereby incorporated by reference in it's entirety.

### FIELD OF THE INVENTION AND INDUSTRIAL APPLICABILITY

The present invention relates to a lentivirus co-culture assay system that can detect modulation of Vif protein activity and that can be formatted for high throughput screening to identify antiviral agents, and a kit for carrying out the method. The present method and kit have industrial applicability in clinical and laboratory methods of evaluating lentiviruses, antiviral agents, and resistance of lentiviruses to antiviral agents.

### BACKGROUND OF THE INVENTION

HIV-1 is a retrovirus of the lentivirus subfamily. The lentiviruses are exogenous, nononcogenic retroviruses causing persistent (chronic active) infections leading to diseases with long incubation periods. These viruses usually infect cells of the immune system (macrophages, T cells) and cause cytopathic effects in permissive cells, such as syncytia and cell death. Lentiviral infections are not cleared by the immune system in most cases, leading to accumulated damage over a period of many years. This important characteristic is reflected in the name of the subfamily (lenti for slow).

Many assays can be used for evaluating the potential antiviral activities of putative lentivirus inhibitors. Traditional antiviral assay methods include quantitatively measuring the production of lentiviral antigens (e.g., HIV-1 p24) or the activities of lentiviral enzymes (e.g., HIV-1 reverse transcriptase) as indicators of virus replication. Although sensitive, these methods are often cumbersome and difficult to format for high throughput screening.

Virus replication can be measured indirectly by monitoring viral induced host-cell cytopathic effects using dye reduction methods (cytopathic effect inhibition assays), which are simple and can usually be adapted for medium-to-high throughput analyses. However, cytopathic effect inhibition assays are limited to highly lytic virus replication systems and often require long assays (>4 days).

Virus replication can also be measured directly or indirectly using reporter gene-based viral replication assays. Such assays can employ a virus or a cell including the reporter gene. Virus replication is quantified by measuring expression of the reporter gene by the virus or cell. Existing reporter gene-based viral replication assays can be limited to particular cells and viral targets.

One target whose function is not effectively tested by many reporter gene-based viral replication assays is the lentivirus Vif protein. HIV-1 viral infectivity factor (Vif) is one of several regulatory proteins encoded by HIV-1. While Vif is required for HIV-1 replication in primary T-cells and some T-cell lines, its precise function is not fully defined. Vif (virion infectivity factor) is a late gene product encoded by most lentiviruses. HIV variants containing mutations in Vif have been observed to replicate at significantly lower levels compared to wild-type virus. Recent studies have shown that Vif counteracts suppression of HIV replication mediated by a host protein known as CEM15 or APOBEC3G, which is a member of the APOBEC cytodine deaminase family of enzymes. In the absence of Vif, APOBEC3G is incorporated into virions during the late stages of virion maturation in virus producing cells. After infection of target cells, APOBEC3G mediates G to A substitustion mutations in newly synthesized HIV-1 cDNA, resulting in a significant accumulation of mutations that ultimately leads to error castotrophy and a reduction in replication competent viral DNA. The interaction of Vif with APOBEC3G in the virus producing cell prevents APOBEC3G from being incorporated into virions and thus prevents APOBEC3G from acting on newly synthesized HIV-1 cDNA. Since APOBEC3G expression and thus this effect is cell-type specific, cell lines may be designated as “permissive” or “non-permissive” for HIV replication, based upon the requirement of Vif for viral replication. For instance, non-permissive cells require a functional Vif to complete the HIV replication cycle. Such cells include primary T-lymphocytes, certain T-cell lines, and macrophages. In contrast, cells which are permissive for HIV replication do not require the presence of a functional Vif protein for HIV replication. Accordingly, agents directed against Vif are likely to interfere with or inhibit viral replication. Since virion-associated Vif is functionally important and is required for viral infectivity in clinically relevent target cells, it is thus an early target for viral inhibition. However, despite recent progress in the field, the precise mechanism of Vif-mediated enhancement of virion infectivity remains unknown. It is apparent that Vif is crucial for HIV infection, yet the role of Vif in virus replication is still not fully defined. Consequently, a need exists for cell-based assays that can measure Vif function and that can evaluate inhibitors of lentivirus replication.

### SUMMARY OF THE INVENTION

The present invention relates to a lentivirus (e.g., HIV) co-culture assay system that can detect modulation of Vif protein activity and that can be formatted for high throughput screening to identify antiviral agents. In one embodiment, this system can be employed for rapid and quantitative measurement of the antiviral activities of test compounds, for example, in dose response assays. In another embodiment, the co-culture assay system can be employed for determining the susceptibility of drug resistant viruses to inhibitors of all classes in a single assay format.

In still another embodiment, the present assay method includes infecting target cells (e.g., T-cells) with lentivirus (e.g., HIV) and co-culturing the infected target cells in the presence of a separate indicator cell.

The target cell can be any cell that can be propagated for at least a short time, including primary cells. Suitable target cells include any transformed cell line or primary cell that (1) supports lentivirus replication and (2) can be propagated in tissue culture for the duration of the assay. The target cell need not be a specially modified cell. Suitable target cells include primary cells, such as peripheral blood mononuclear cells, patient derived T-cells, or macrophages. In one embodiment, the target cells include cell lines such as MT-2, H9, PM1, CEM-SS, MT-4, CEM, Hut78, Sup-T1, HSB-2, JM, 174XCEM, Jurkat, Molt-4, VB, or WE17/10 T-cells; HeLa T4 or derivatives thereof; or Hos-CD4 cells. Preferably, the target cells include cells that may exhibit a Vif-nonpermissive phenotype, such as PM-1, MT-2, H9, Hut78, 174XCEM, C8166, MT-4, or Jurkat cells. Preferred Vif-nonpermissive cell for use in the present invention include PM-1, MT-2, H9, or Hut78 cells.

The indicator cell can be any cell permissive to lentivirus infection and containing a reporter gene that can be induced after infection of the indicator cell by virus produced from the infected target cells. In one embodiment, the indicator cells are adherent and highly permissive to single-round HIV-1 infections, yet the indicator cells do not support high levels of virus amplification. Examples include HeLa cells expressing CD4 (e.g., HeLa CD4 LTR/lacZ cells). Other suitable indicator cells include cells similar to HeLa CD4 LTR/lacZ cells (e.g., MAGI-CCR5 or TMZ-bl cells), or other engineered indicator cell lines, such as U373-MAGI, CEM-GFP, HOS-Ghost cells, or Jurkat 1G5 cells. In a preferred embodiment, the target cells include HeLa CD4 LTR/lacZ cells or MAGI-CCR5 cells. Such cells are known and/or commercially available.

The indicator cell also preferably contains a reporter gene induced after infection of the indicator cell by virus produced from the infected target cells. Expression of the reporter gene can be under the control of a promoter or other sequences that are either directly or indirectly responsive to virus infection. Indicator cells can contain a reporter that is either activated or suppressed by viral infection. For example, the reporter gene can be under the control of a lentivirus promoter, such as HIV LTR. Suitable promoters for virus induced reporter expression include HIV LTR (e.g., HIV-1 LTR or HIV-2 LTR), SIV LTR, EIAV (equine infectious anemia virus) LTR, or HTLV-1 LTR. In a preferred embodiment, the promoter includes HIV-1 LTR or HIV-2 LTR. The reporter can be introduced either stably or transiently by DNA or RNA transfection of the indicator cell or by using viral vectors. Such promoters are known and/or commercial available.

Suitable reporter genes encode detectable proteins, such as proteins that can produce color, light, fluorescence, or the like. In one embodiment, the detectable protein is selected from a group that includes β-galactosidase, luciferase (e.g., Photinus pyralis luciferase (firefly luciferase), Renilla reniformis luciferase (Renilla luciferase)), secreted alkaline phosphatase, fluorescent protein (e.g., Aequora victoria green fluorescent protein (GFP), blue fluorescent protein, yellow fluorescent protein, and cyan fluorescent protein), or β-lactamase reporter. Genes encoding such proteins, methods for detecting such proteins, and kits for using such proteins are known and/or commercially available.

In one embodiment, the indicator cells include HeLa CD4 LTR/lacZ cells.

In another embodiment, the present invention includes a co-culture method for detecting replication of a lentivirus. This method can include infecting Vif-nonpermissive cells with the lentivirus in vitro; co-culturing the infected Vif-nonpermissive cells and indicator cells; contacting the co-culture with a test compound; and assaying for the indicator cells in the co-culture.

In a further embodiment, infecting includes incubating Vif-nonpermissive cell with the lentivirus for one or more hours and washing the Vif-nonpermissive cells.

In another embodiment, infecting includes infecting the Vif-nonpermissive cells with lentivirus at a multiplicity of infection of less than 0.2. In an embodiment, infecting includes infecting the Vif-nonpermissive cells with lentivirus at a multiplicity of infection of greater than 0.2.

In still another embodiment, the method also includes incubating indicator cells in culture container for one or more hours before co-culturing.

In a further embodiment, contacting the co-culture with a test compound includes adding the test compound to the indicator cells before co-culturing. In an embodiment, contacting the co-culture with a test compound includes adding the test compound to the Vif-nonpermissive cells before co-culturing.

In another embodiment, assaying for the indicator includes adding an enzyme substrate to the co-culture. In an embodiment, assaying for the indicator includes assaying for chemiluminescence. In an embodiment, assaying for the indicator includes lysing cells of the co-culture.

In another embodiment, the present invention includes a method for detecting an inhibitor of activity of lentivirus Vif. This embodiment of the method can include infecting Vif-nonpermissive cells with the lentivirus in vitro; co-culturing the infected Vif-nonpermissive cells and indicator cells; contacting the co-culture with a test compound; assaying for the indicator in the co-culture; wherein indicator below a certain threshold level indicates inhibition of lentivirus replication by the test compound and that the test compound is a lentivirus replication inhibitor; and challenging a Vif independent lentivirus replication assay with the lentivirus replication inhibitor; lentivirus replication above a certain threshold level indicating inhibition of Vif-activity by the lentivirus replication inhibitor and that the lentivirus replication inhibitor is the inhibitor of activity of lentivirus Vif.

In another embodiment, challenging a Vif independent lentivirus replication assay with the lentivirus replication inhibitor can include assaying the lentivirus replication inhibitor against the lentivirus in a Vif-permissive cell.

In a further embodiment, challenging a Vif independent lentivirus replication assay with the lentivirus replication inhibitor can include assaying the lentivirus replication inhibitor against a replication competent lentivirus lacking a functional Vif-gene.

In still another embodiment, the replication competent lentivirus lacking a functional Vif-gene includes: a lentivirus analogous to a lentivirus that includes a functional Vif-gene; a lentivirus that is a different strain of a lentivirus that includes a functional Vif-gene; a lentivirus related to a lentivirus that includes a functional Vif-gene; or a mixture or combination thereof.

In another embodiment, the present invention includes a method for indicating replication of HIV. This embodiment of the method can include adding HeLa CD4 LTR/lacZ indicator cells to a vessel; adding test compound to the vessel; contacting MT-2 or PM1 Vif-nonpermissive cells with HIV; incubating the Vif-nonpermissive cells and HIV for about 1 to about 4 hours; washing the incubated Vif-nonpermissive cells with cell culture medium; adding the washed Vif-nonpermissive cells to the vessel containing the indicator cells to form a mixture of Vif-nonpermissive cells, indicator cells, and test compound; co-culturing the mixture for about 1 to about 8 days; and monitoring the culture for β-galactosidase activity; wherein the level of β-galactosidase activity indicates the level of HIV in the culture.

In a further embodiment, the present invention includes a method for detecting an inhibitor of activity of HIV Vif. This embodiment of the method includes adding HeLa CD4 LTR/lacZ indicator cells to a vessel; adding test compound to the vessel; contacting MT-2 or PM1 Vif-nonpermissive cells with HIV; incubating the Vif-nonpermissive cells and HIV for about 1 to about 4 hours; washing the incubated Vif-nonpermissive cells with cell culture medium; adding the washed Vif-nonpermissive cells to the vessel containing the indicator cells to form a mixture of Vif-nonpermissive cells, indicator cells and test compound; co-culturing the mixture for about 1 to about 8 days; and monitoring the culture for β-galactosidase activity; wherein activity below a threshold level indicates inhibition of HIV replication by the test compound and that the test compound is lentivirus replication inhibitor; and challenging a Vif independent lentivirus replication assay with the lentivirus replication inhibitor; lentivirus replication above a second threshold level indicating inhibition of Vif-activity by the lentivirus replication inhibitor and that the lentivirus replication inhibitor is the inhibitor of activity of lentivirus Vif. In an embodiment, challenging a Vif independent lentivirus replication assay with the lentivirus replication inhibitor includes assaying the lentivirus replication inhibitor against the lentivirus in a Vif-permissive cell. In an embodiment, challenging a Vif independent lentivirus replication assay with the lentivirus replication inhibitor can include assaying the lentivirus replication inhibitor against a replication competent lentivirus lacking a functional Vif-gene.

In still another embodiment, the lentivirus includes human immunodeficiency virus (HIV), simian immunodeficiency virus (SIV), simian AIDS retrovirus SRV-1, feline immunodeficiency virus (FIV), Caprine arthritis encephalitis virus (CAEV), Bovine immunodeficiency virus (BIV), and Visna/maedi virus, and the like. In a preferred embodiment, the lentivirus includes HIV-1 or HIV-2. In an embodiment, the lentivirus includes HIV-1. In a more preferred embodiment, the HIV includes a clinical isolate or a laboratory strain.

In yet another embodiment, a kit is provided for carrying out the present co-culture assay. Such a kit can include, for example, target cells and indicator cells, plus one or more of buffers or medium for working with the cells, indicator reagent (e.g., substrate for an indicator enzyme), instructions for using the kit, packaging containing the kit, representative standard curves, and the like. The kit can optionally also include test compound standards, lentivirus standards, or other standards useful for calibrating or using the kit.

### BRIEF DESCRIPTION OF THE FIGURES

FIG. 1 schematically illustrates an embodiment of the present lentivirus co-culture assay adapted to HIV.

FIG. 2 illustrates results of experiments that demonstrated inhibition of replication of HIV-1 NL4.3 in an HIV MT-2 cell co-culture assay. Reporter gene activity is presented as relative light units (RLU). These experiments demonstrated that inhibitors active both early (a non-nucleoside reverse transcriptase inhibitor) or late (a protease inhibitor) in the HIV life cycle were detected in this co-culture assay. The reporter signal measured was dependent on HIV-1 replication.

FIG. 3 illustrates results of experiments that demonstrated inhibition of replication of HIV-1 NL4.3 in the HIV PM1 cell co-culture assay. Reporter gene activity is presented as relative light units (RLU). These experiments demonstrated that inhibitors active both early (a non-nucleoside reverse transcriptase inhibitor) or late (a protease inhibitor) in the HIV life cycle were detected in this co-culture assay. The reporter signal measured was dependent on HIV-1 replication.

FIG. 4 illustrates results of experiments that demonstrated inhibition of replication of HIV-1 NL4.3 in an assay measuring replication directly in the HeLa CD4 LTR/lacZ indicator cells. Reporter gene activity is presented as relative light units (RLU). These experiments demonstrated that inhibitors active early (a non-nucleoside reverse transcriptase inhibitor) in the HIV life cycle were detected in this direct reporter cell assay. However, inhibitors active late (a protease inhibitor) in the HIV life cycle were not adequately detected in this direct reporter cell assay.

FIG. 5 illustrates results of experiments that demonstrated that MT-2 T cells are nonpermissive for HIV Vif. A virus including Vif replicated at high levels in these cells. A virus encoding a defective Vif replicated only poorly in these cells. HIV replication was detected by p24 production in the T cells.

FIG. 6 illustrates results of experiments that demonstrated that PM1 T cells are nonpermissive for HIV Vif. A virus including Vif replicated at high levels in these cells. A virus encoding a defective Vif replicated only poorly in these cells. HIV replication was detected by p24 production in the T cells.

FIG. 7 illustrates results of experiments that demonstrated that replication in the MT-2 or PM1 cell-based co-culture assays is dependent on a functional Vif gene. Reporter gene activity is presented as relative light units (RLU).

FIG. 8 illustrates the results of experiments that demonstrated the proportion of hits found among known HIV replication inhibitors when tested at EC50 and EC90 in the MT-2 cell-based co-culture assay formatted as a high throughput screen.

### DETAILED DESCRIPTION OF THE INVENTION

In accordance with the present invention there may be employed conventional molecular biology, microbiology, and recombinant DNA techniques within the skill of the art. Such techniques are explained fully in the literature. See, e.g., Maniatis et al., “Molecular Cloning: A Laboratory Manual,” (1989); Ausubel, Ed., “Current Protocols in Molecular Biology,” Volumes I-III (1994); Celis, Ed., “Cell Biology: A Laboratory Handbook,” Volumes I-III (1994); Coligan, Ed., “Current Protocols in Immunology,” Volumes I-III (1994); Gait, Ed., “Oligonucleotide Synthesis” (1984); Hames et al., Eds., “Nucleic Acid Hybridization” (1985); Hames et al., “Transcription and Translation” (1984); Freshney, Ed., “Animal Cell Culture” (1986); IRL Press, “Immobilized Cells and Enzymes” (1986); and Perbal, “A Practical Guide To Molecular Cloning” (1984).

Definitions and Abbreviations

As used herein, the terms “comprising” and “including” are used in an open, non-limiting sense.

As used herein, the term “Vif-nonpermissive” refers to cells that require the virus to encode an active Vif for the cells to support viral replication. Cells that do not require Vif expression to support lentivirus replication are referred to as “Vif-permissive”.

As used herein, the terms “co-culture” and “culture” refer to cells in conditions in which they can grow, multiply, and/or support lentivirus replication. These terms exclude cells that have been fixed. A culture or co-culture can include or be processed into cells that have been lysed, for example, for using or detecting an indicator gene or protein.

As used herein, the phrase “Vif independent lentivirus replication assay” refers to an assay system in which lentivirus replication does not require an active Vif.

As used herein, the term “replicon” refers to any genetic element (e.g., plasmid, chromosome, viral RNA) that functions as an autonomous unit of DNA or RNA replication in vivo. That is, it is capable of replication under its own control. Bradenbeck et al., Semin. Virol. 3:297-310 (1992).

As used herein, the term “multiplicity of infection” (MOI) refers to the number of infectious units (IU) per cell in culture.

As used herein, the term “vector” refers to a circular DNA, such as a plasmid, phage or cosmid, to which another DNA segment may be attached so as to bring about the replication, expression or integration of the attached segment.

A variety of expression vectors can be used to express a nucleic acid molecule. Such vectors include chromosomal, episomal, and virus-derived vectors, e.g., vectors derived from bacterial plasmids, from bacteriophage, from yeast episomes, from yeast chromosomal elements, including yeast artificial chromosomes, from viruses such as baculoviruses, papovaviruses such as SV40, vaccinia viruses, adenoviruses, poxviruses, pseudorabies viruses, herpes viruses, and retroviruses. Vectors may also be derived from combinations of these sources, such as those derived from plasmid and bacteriophage genetic elements, e.g., cosmids and phagemids. Appropriate cloning and expression vectors for prokaryotic and eukaryotic hosts are described in Sambrook et al., supra.

A vector containing the appropriate nucleic acid molecule can be introduced into an appropriate cell for propagation or expression using known techniques. Cells for vector propagation can include bacterial cells including, but not limited to, E. coli, Streptomyces, and Salmonella typhimurium, eukaryotic cells including, but not limited to, yeast, insect cells, such as Drosophila, animal cells, such as Huh-7, HeLa, COS, HEK 293, MT-2, CEM-SS, and CHO cells, and plant cells.

Vectors generally include selectable markers that enable the selection of a subpopulation of cells that contain the recombinant vector constructs. The marker can be contained in the same vector that contains the nucleic acid molecules described herein or may be on a separate vector. Markers include tetracycline- or ampicillin-resistance genes for prokaryotic host cells and dihydrofolate reductase or neomycin resistance for eukaryotic host cells. However, any marker that provides selection for a phenotypic trait will be effective.

In the case of bacteriophage and viral vectors, these can be introduced into cells as packaged or encapsulated virus by standard procedures for infection and transduction. Viral vectors can be replication-competent or replication-defective. In the case in which viral replication is defective, replication will occur in host cells providing functions that complement the defects.

A cell has been “transformed” by exogenous or heterologous DNA or RNA when such DNA or RNA has been introduced inside the cell. The transforming DNA or RNA may or may not be integrated (covalently linked) into chromosomal DNA making up the genome of the cell. For example, in prokaryotes, yeast, and mammalian cells, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. In the case of an RNA replicon that transforms a mammalian cell as described in the present invention, the RNA molecule, e.g., an HCV RNA molecule, has the ability to replicate semi-autonomously. Huh-7 cells carrying the HCV replicons get selected in the presence of G418 since HCV RNA replication results in resistance to G418 by production of the neomycin phosphotransferase protein. This results in clones of Huh-7 cells resistant to G418, which are capable of forming cell lines. These clones of cells can be further transformed/transduced with expression vectors, such as the one that carries the firefly luciferase gene (pcDNA6.Fluc) to generate stable cell lines that require selection by two antibiotic markers.

As used herein, the term “clone” refers to a population of cells derived from a single cell or common ancestor by mitosis.

As used herein, the term “cell line” refers to a clone of a primary cell that is capable of stable growth in vitro for many generations. RNA or DNA molecules, which can be used to transform or “transfect” cells can be used for making transformed cell lines. For some RNA viruses, such methods can be used to produce cell lines which transiently or continuously support virus replication and, in some cases, which produce infectious viral particles.

As used herein, the term “lentivirus” refers to any one of a family of retroviruses that can infect mammals such as cows, sheep, cats, primates, and the like. Known lentiviruses include human immunodeficiency virus (HIV), simian immunodeficiency virus (SIV), simian AIDS retrovirus SRV-1, feline immunodeficiency virus (FIV), Caprine arthritis encephalitis virus (CAEV), Bovine immunodeficiency virus (BIV), and Visna/maedi virus.

Replication of a lentivirus in cells can be ascertained by branched TaqMan quantitative RT/PCR and immunological procedures. The procedures and their application are well known in the art and accordingly may be utilized within the scope of the present invention. A “competitive” antibody binding procedure is described in U.S. Pat. Nos. 3,654,090 and 3,850,752. A “sandwich” procedure is described in U.S. Pat. Nos. RE 31,006 and 4,016,043. Still other procedures are known such as the “double antibody”, or “DASP” procedure.

In each instance, lentivirus proteins form complexes with one or more antibodies or binding partners and one member of the complex is labeled with a detectable label. The fact that a complex has formed and, if desired, the amount thereof, can be determined by known methods applicable to the detection of labels.

Alternatively, the presence of lentivirus RNA can be determined by Northern analysis, primer extension, and the like. The labels most commonly employed for these studies are radioactive elements, enzymes that fluoresce when exposed to substrate and others. A number of fluorescent materials are known and can be utilized as labels. These include, for example, fluorescein, rhodamine, auramine, Texas Red, AMCA blue and Lucifer Yellow.

An antibody to lentivirus proteins or a probe for lentivirus RNA can also be labeled with a radioactive element or with an enzyme. The radioactive label can be detected by any of the currently available counting procedures. The preferred isotope may be selected from ³H, ¹⁴C, ³²P, ³⁵S, ³⁶Cl, ⁵¹Cr, ⁵⁷Co, ⁵⁸CO, ⁵⁹Fe, ⁹⁰Y, ¹²⁵I, ¹³¹I, and ¹⁸⁶Re.

Enzyme labels are likewise useful, and can be detected by any of the presently utilized colorimetric, spectrophotometric, fluorospectrophotometric, techniques. The enzyme is conjugated to the selected probe by reaction with bridging molecules such as carbodiimides, diisocyanates, glutaraldehyde and the like. Many enzymes that can be used in these procedures are known and can be utilized. Those preferred are peroxidase, beta-glucuronidase, beta-D-glucosidase, beta-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods. In addition, a probe may be biotin-labeled, and thereafter be detected with labeled avidin, or a combination of avidin and a labeled anti-avidin antibody. Probes may also have digoxygenin incorporated therein and be then detected with a labeled anti-digoxygenin antibody.

An EC₅₀ value is the concentration of the inhibitor at which 50% inhibition of viral replication is achieved. A co-culture lentivirus assay system can be developed to determine the specific antiviral activity of inhibitors in standard dose response assays. In such assays, the co-culture assay can be conducted in 96 well microtiter plates containing serial dilutions of test inhibitors or no inhibitor. Data from the reporter gene measurements can be expressed as the percent of reporter gene activity in inhibitor-treated cells relative to that of inhibitor-free cells. An analysis of the antiviral component of such data allows for the calculation of the fifty-percent effective concentration (EC₅₀). Similarly, an EC₉₀ value is the concentration of the inhibitor at which 90% inhibition of viral replication is achieved, and an analysis of the antiviral component of a data set allows for a calculation of the ninety-percent effective concentration (EC₉₀).

The abbreviations used herein include: “3TC” means lamivudine; “ADV” means adefovir dipivoxil; “ATA” means aurintricarboxylic acid; “AZT” means 3′-Azido-3′-deoxythymidine; “CPV” means Capravirine; “d4T” means stavudine; “ddl” means didanosine; “DLV” means delavirdine; “DMEM” means Dulbeccos's Modified Eagle Medium; “DMSO” means dimethyl sulphoxide; “EC₉₀” means 90% effective concentration of inhibitor or drug; “EC₅₀” means 50% effective concentration of inhibitor or drug; “EFV” means efavirenz; “FBS” means fetal bovine serum; “IDV” means indinavir; “HEK” means human embryonic kidney; “IN” means integrase; “INI” means integrase inhibitor; “I” means liter; “mg” means milligram; “ml” means milliliter; “moi” or “MOI” means multiplicity of infection; “NFV” means nelfinavir; “ng” means nanogram, “NNRTI” means non-nucleoside reverse transcriptase inhibitor; “NRTI” means nucleoside analog reverse transcriptase inhibitor; “NVP” means Nevirapine; “PCR” means polymerase chain reaction; “PI” means protease inhibitor; “RLU” means relative light units; “RPMI” means RPMI medium 1640; “RT” means reverse transcriptase; “SD” means standard deviation; “SQV” means saquinavir; “TCID₅₀” means 50% tissue culture infectious dose; “ug” or “μg” mean microgram; “ul” or “μl” means microliter; “M” or “μM” means micromolar.

Co-Culture Assay System

The present invention relates to a lentivirus (e.g., HIV) co-culture assay system that can detect modulation of Vif protein activity and that can be formatted for high throughput screening to identify antiviral agents. The present co-culture assay offers several advantages over known reporter virus and reporter cell assays. The present assay method includes infecting target cells (e.g., T-cells) with lentivirus (e.g., HIV) and co-culturing the infected target cells in the presence of a separate indicator cell. This co-culture assay system can employ any target cell that can be propagated for at least a short time, including primary cells. The target cell need not be a specially modified cell. The indicator cell is permissive to lentivirus infection and contains a reporter gene induced after infection of the indicator cell by virus produced from the infected target cells. For example, the reporter gene can be under the control of a lentivirus promoter, such as HIV LTR. Preferably, the assay is designed such that the indicator cells are highly permissive to single-round infections but do not support high levels of virus amplification.

Virus amplification can occur primarily in the unmodified target cells rather than the indicator cells. Virus replication is then measured by monitoring reporter gene activation in the indicator cells, which results from periodic infection of the indicator cells by virus replicating in the target cells. In an embodiment, one round of lentivirus replication in the target cells generates a detectable signal in the indicator cells. Multiple rounds of replication in the target cells can generate maximal reporter gene signals.

This system provides a single assay format that can detect suppression of reporter gene signals by inhibitors of early and/or late stage inhibitors of lentivirus replication. The co-culture assay can include all of the HIV targets required for replication in T-cells, including HIV targets that are required in primary T-cells but not necessarily all T cell lines. Quantitation of the reporter gene data for a series of concentrations of an inhibitor provides a direct calculation of EC₉₀ values for lentivirus inhibitors of all classes. In embodiments, the assay can be employed in low-, medium-, or high-throughput formats, and can be used in HIV drug discovery activities.

The present co-culture assay format preferably includes HIV-1 Vif as an antiviral target. In an embodiment, the system employs Vif non-permissive target cells. For example, the system can employ cells that may exhibit a Vif non-permissive phenotype, such as PM-1, MT-2, H9, Hut78, 174XCEM, C8166, MT-4, or Jurkat cells. In an embodiment, such cells include PM-1, MT-2, H9, or Hut78 cells. In an embodiment, the method can employ HIV-1 Vif non-permissive T-cells (i.e., T-cells that require that the virus encode active Vif for viral replication). Such T-cell lines include MT-2 and PM1. Inhibitors of Vif activity can be detected in a high throughput format antiviral assay.

In an embodiment, the co-culture assay can determine dose-response curves for inhibitors. For determining dose response curves, target cells are infected with virus. At a specified time after infection, infected target cells can be added to microtiter (e.g., 96-well) plates containing indicator cells and serial dilutions of test inhibitors or no inhibitor. At a specified time after co-culture of the infected target cells with indicator cells, the activity of the reporter gene present in the indicator cells can be measured using the appropriate reporter gene assay methods. Data from the reporter gene measurements can be expressed as the percent of reporter gene activity in infected inhibitor-treated cells relative to that of infected, inhibitor-free cells. An analysis of such data allows for the calculation of the fifty percent effective concentration (EC₅₀) or ninety percent effective concentration (EC₉₀) of an inhibitor.

In an embodiment, the co-culture assay can be used to screen for specific antiviral inhibitors in a high throughput format. In a high throughput screen format, putative inhibitors can be added at single or multiple doses to indicator cells in microtiter plates. Target cells can be infected for a specified time and then added to the wells containing indicator cells and inhibitor or no inhibitor control wells. At a specified time after co-culture of the infected target cells with indicator cells, the activity of the reporter gene present in the indicator cells can be measured using the appropriate reporter gene assay methods. Data from the reporter gene measurements can then be expressed as the percent inhibition of reporter gene activity in infected inhibitor-treated cells relative to that of infected inhibitor-free cells. Antiviral activity can then be assigned to test inhibitors that effect a significant reduction in the reporter gene activity relative to the no compound control wells.

The present assay system can be demonstrated to be amenable to use in a high-throughput format. For example, coefficients of variation and screening window coefficients (Z′ value) can be determined for the present assay system. The Z′ value is reflective of the dynamic range as well as the variation of the assay and is a useful tool for assay comparisons and assay quality determinations (Zhang et al., J. Biomolec. Screen 4:67-73 (1999)). Typically a Z′ value >0.5 is considered favorable for high-throughput screening.

In an embodiment, the co-culture assay can be used to determine the susceptibility of drug resistant viruses to HIV inhibitors of all classes in a single assay format. In such susceptibility assays, target cells are infected with either wild-type (wt) virus or virus variants containing drug resistant mutations of interest. At a specified time after infection, either wt or drug resistant virus infected target cells can be added to microtiter (e.g., 96-well) plates containing indicator cells and serial dilutions of test inhibitors or no inhibitor. At a specified time after co-culture of the infected target cells with indicator cells, the activity of the reporter gene present in the indicator cells can be measured using the appropriate reporter gene assay methods. Data from the reporter gene measurements can be expressed as the percent of reporter gene activity in infected inhibitor-treated cells relative to that of infected, inhibitor-free cells. An analysis of such data would allow for the calculation of the fifty percent effective concentration (EC₅₀) or ninety percent effective concentration (EC₉₀) of an inhibitor. Furthermore, the fold-change in antiviral activity, which is a measure of the resistance to a particular inhibitor conferred by mutations present in viral variants, could be calculated by dividing the EC₅₀ or EC₉₀ of an inhibitor determined for the drug resistant virus by the EC₅₀ or EC₉₀ of an inhibitor determined for the wt virus.

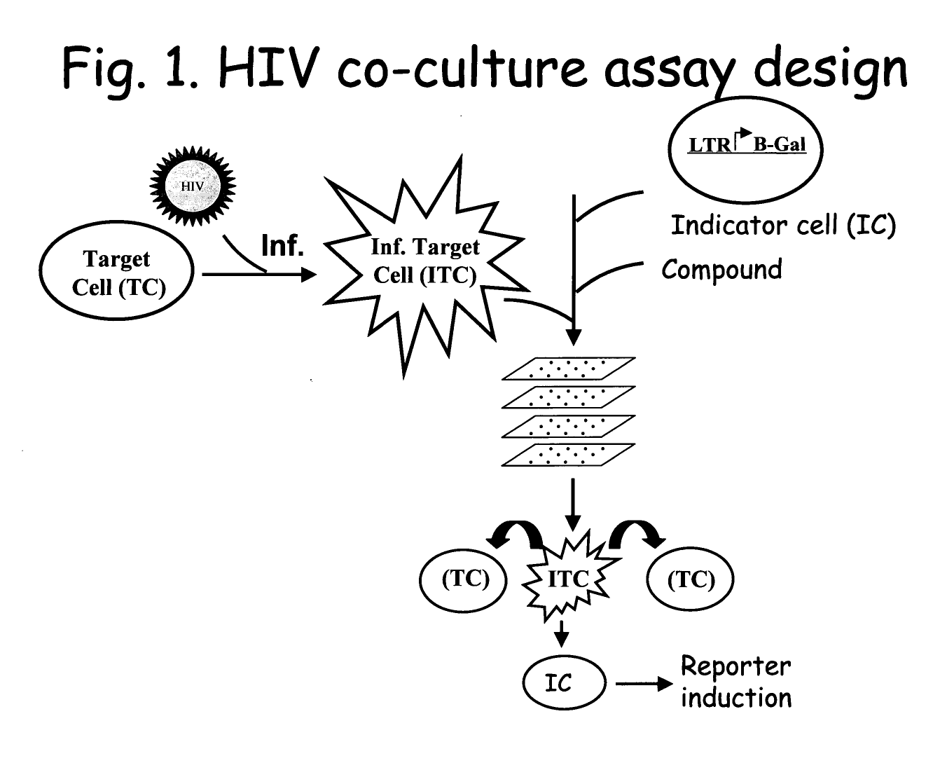

FIG. 1 schematically illustrates an embodiment of the present co-culture assay method as it can be applied to HIV. In this illustrated embodiment, target cells (e.g., T-cells) are infected with virus for a specified period of time. Virus infected target cells are then washed and added to microtiter plates containing a separate indicator cell line. In this illustrated embodiment, the indicator cell line is HeLa CD4 LTR/lacZ cells. The microtiter plates can also contain test compound or vehicle control. The indicator cell line is permissive to HIV infection and contains a reporter gene (e.g., β-galactosidase) that is induced after infection of the indicator cell by virus produced from the infected target cells. Three or four days after infection, virus replication is measured by quantifying reporter gene activity in the indicator cell line.

The co-culture assay system of this invention can be used for any lentivirus or replication competent lentivirus derivative. By replication competent virus derivative, we mean any viral replicon containing viral sequences that encode the potential to infect cells and direct replication of either full-length or subgenomic viral cDNAs via viral promoters and/or replication signal sequences. Alternatively, replication competent virus derivatives could contain a portion of the viral sequences required to encode the potential to infect cells and replicate with the remaining required components provided in trans in the target cells. In addition, the co-culture assay can be used for any infectious virus generated through homologous recombination events in the infected target cell.

The present invention may be better understood with reference to the following examples. These examples are intended to be representative of specific embodiments of the invention, and are not intended as limiting the scope of the invention.

### EXAMPLES

Materials

The following materials were employed in the Examples.

Cells

HeLaCD4 LTR/lacZ, MT-2, PM1, and HEK 293 cells were obtained through the National Institutes of Health AIDS Research and Reference Reagent Program, Bethesda, MD. HeLaCD4 LTR/lacZ cells and HEK293 cells were propagated in Dulbeccos's Modified Eagle Medium (Life Technologies, Gaithersburg Md.) containing 10% fetal bovine serum (HyClone, Logan, Utah). MT-2 cells and PM1 cells were propagated in RPMI medium 1640 (Life Technologies, Gaithersburg Md.) containing 10% FBS (HyClone).

Plasmids and Virus

The HIV-1 infectious cDNA, pNL4.3 (Accession No. AF324493) was obtained through the National Institutes of Health AIDS Research and Reference Reagent Program, Bethesda, Md.

To construct the NL4.3 Vif mutant virus (HIV-1 NL4.3/ΔVif), a 169-nucleotide deletion was introduced in the Vif coding region of pNL4.3 (nucleotide positions 5151-5320) using polymerase chain reaction based mutagenesis (Horton et al., BioTechniques 8:528-535 (1990)). The resulting pHIV-1 NL4.3/ΔVif cDNA encoded a 56 amino acid deletion in Vif sequences.

To generate infectious virus, pNL4.3 or pHIV-1 NL4.3/ΔVif was transfected into HEK 293 cells using the LipofectAMINE Plus transfection kit according to the manufacturers protocol (Life Technologies). 72 hours after transfection, infectious HIV-1 NL4.3 wt virus or HIV-1 NL4.3/ΔVif mutant virus was harvested from the supernatants of transfected cells and clarified by centrifugation (500×g). Titers (TCID₅₀) of the resulting viral stocks were determined after infecting HeLaCD4 LTR/lacZ target cell lines with serial dilutions of the viral stocks (Johnson and Byrington, 1990) and measuring beta-galactosidase (β-Gal) activity in the HeLaCD4 LTR/lacZ 72 hours after infection using a reporter gene assay kit (Dual-Light™ System; Chemiluminescent Reporter Gene Assay System for the Combined Detection of Luciferase and β-Galactosidase, Applied Biosystems, Bedford Mass.).

Compounds

Capravirine (CPV) was synthesized at Agouron Pharmaceuticals Inc. (San Diego, Calif.). NFV, L-870810 and S-1360 were synthesized at Agouron Pharmaceuticals Inc. (San Diego, Calif.). Nevirapine (NVP), saquinavir (SQV), efavirenz (EFV), and indinavir (IDV) were obtained through the National Institutes of Health AIDS Research and Reference Reagent Program, Bethesda, Md. Delavirdine (DLV), lamivudine (3TC), and stavudine (d4T) were kindly provided by Pharmacia and Upjohn (Kalamazoo, Mich.), Glaxo Wellcome (Research Triangle Park, N.C.), and Bristol-Myers Squibb (Wallingford, Conn.), respectively. Adefovir dipivoxil (ADV) was synthesized and purchased from Pharm-Eco (Devens, Mass.). 3′-Azido-3′-deoxythymidine (AZT), Aurintricarboxylic acid (ATA) and didanosine (ddl) were purchased from Sigma-Aldrich (St Louis, Mo.).

### Example 1

### MT-2 Cell-Based Co-culture Assay Detects HIV-1 Replication and its Inhibition

MT-2 cells were successfully utilized in the HIV co-culture assay format.

HeLa CD4 LTR/lacZ indicator cells were added to 96-well microtiter plates at cell densities of 1×10⁴ cells/well in DMEM or RPMI medium (Life Technologies) containing 10% FBS (HyClone). MT-2 cells were infected with HIV-1 NL4.3 using 656, 1312, 2624, or 5248 TCID₅₀s per 1.6×10⁴ cells. Two hours after infection, infected MT-2 cells were washed with RPMI medium (Life Technologies) and added to the 96-well microtiter plates containing the HeLa CD4 LTR/lacZ indicator cells. The final MT2-cell densities in the indicator wells were 1.6×10⁴ cells/well. Certain of the wells with indicator cells also included either non-nucleoside reverse transcriptase inhibitor EFV or protease inhibitor NFV at final concentrations of 0.1 uM or 1 uM, respectively.

Virus replication was measured 4 days after infection by quantifying HIV-1 Tat induced beta-galactosidase (β-Gal) activity in the HeLa CD4 LTR/lacZ indicator cells using the Dual-Light™ System according to the manufacturer's protocol (Applied Biosystems). Experiments were performed in replicates of 3 or more.

The results showed a significant induction of β-Gal activity in the co-culture assay format, which was dependent on viral input (TCID₅₀). At the lowest virus TCID₅₀ (656), a 244-fold induction of reporter gene signal was observed in the co-culture assay, with a maximum signal of ˜520,000 relative light units. Therefore, the reporter signal measured in the MT-2 cell-based HIV co-culture assay is sufficient for high throughput screening in microtiter plates even at the lowest viral input tested.

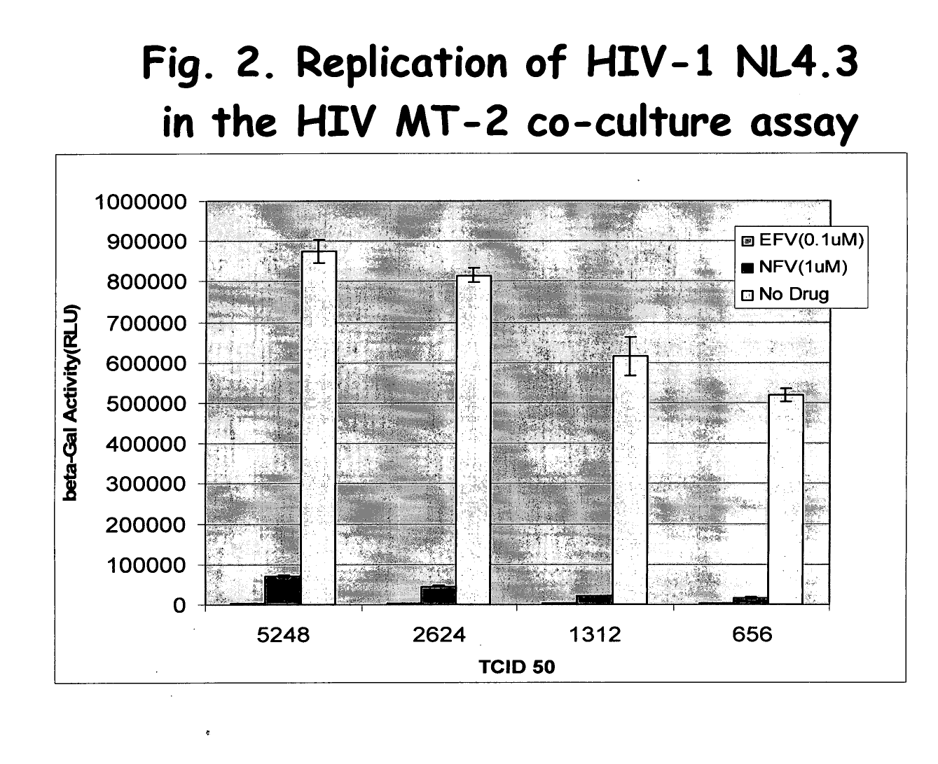

To further demonstrate that the reporter gene signal was dependent on virus infection, infections were performed in the presence of a non-nucleoside reverse transcriptase inhibitor. The results showed a >99% inhibition of the reporter signal when cells were infected in the presence of the NNRTI efavirenz at concentrations that are >10-fold higher than the EC₉₀ of this drug (0.1 uM) (FIG. 2).

To determine whether the reporter gene signal was dependent on multiple rounds of virus replication rather than a single round of infection, infections were performed in the presence of a protease inhibitor. HIV-1 protease inhibitors act during the late stages of infection (infectious virion production) and therefore do not inhibit the initial round of infection in tissue culture. As shown in FIG. 2, about 98% of the reporter signal was inhibited when cells were infected in the presence of the PI nelfinavir at 10×EC₉₀ concentrations.

These results demonstrate that the reporter signal in the MT-2 cell-based HIV co-culture assay were dependent on multiple rounds of virus replication. Further, these results indicate that the MT-2 cell-based HIV co-culture assay can (and did) detect inhibitors that target any step in the HIV-1 replication cycle.

### Example 2

### PM1 Cell-Based Co-Culture Assay Detects HIV-1 Replication and its Inhibition

PM1 cells were successfully utilized in the HIV co-culture assay format.

These experiments were conducted by a modification of the method employed in Example 1. The modifications were as follows: PM1 cells were used in place of the MT-2 cells. PM1 cells were infected with HIV-1 NL4.3 using 656, 1312, 2624, or 5248 TCID₅₀s per 2×10⁴ cells.

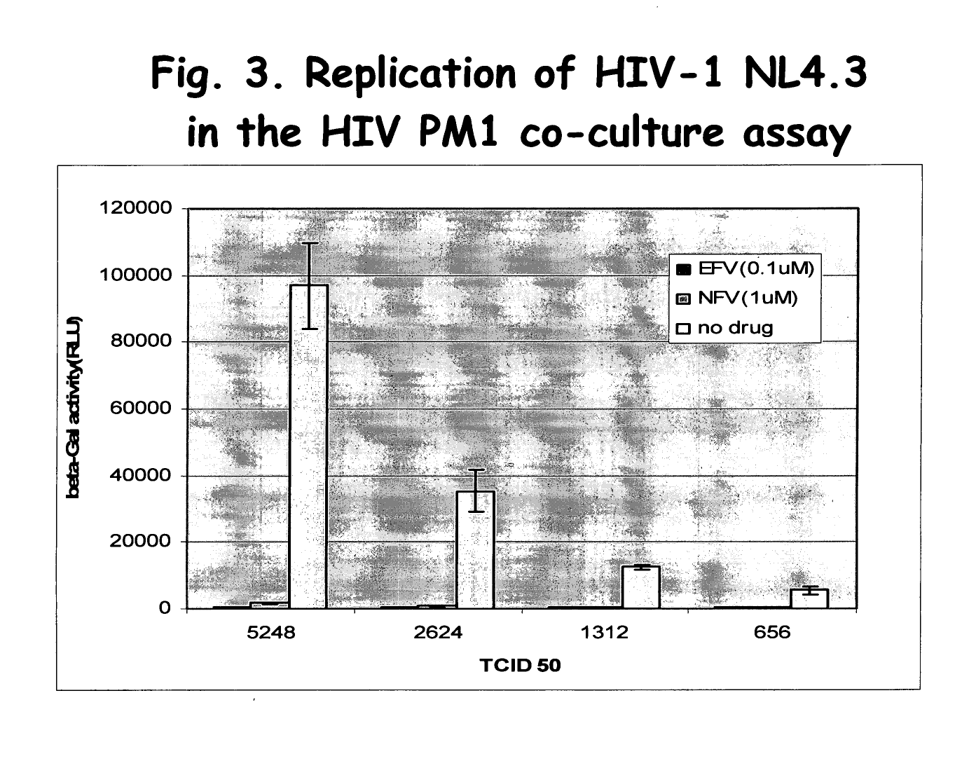

The results showed a significant induction of β-Gal activity in the co-culture assay format, which was dependent on viral input (TCID₅₀). At the highest virus TCID₅₀ (5248), a 265-fold induction of reporter gene signal was observed in the co-culture assay, with a maximum signal of ˜97,000 relative light units (FIG. 3). In addition, >99% of the reporter signal was inhibited by ≧10×EC₉₀ concentrations of the non-nucleoside reverse transcriptase inhibitor EFV or the protease inhibitor NFV in the co-culture assay (FIG. 3).

These results demonstrate that the PM1 cell-based HIV co-culture assay produced a reporter signal sufficient for high throughput screening in microtiter plates. In addition, the reporter signal in the PM-1 cell-based HIV co-culture assay was dependent on multiple rounds of virus replication, as demonstrated by 99% inhibition of the reporter signal in the presence of the protease inhibitor NFV.

### Example 3

### Direct Infection HeLa CD4 LTR/lacZ Indicator Cells by HIV-1

This Example compared the present HIV co-culture assay with a known reporter cell-based assay method. This known method involved direct infection of HeLa CD4 LTR/lacZ indicator cells with HIV-1 (Kimpton and Emerman, J. Virol., 66(4):2232-2239 (1992)).

HeLa CD4 LTR/lacZ indicator cells were directly infected with TCID₅₀s identical to those used in the co-culture experiments described in Examples 1 and 2. HeLa CD4 LTR/lacZ indicator cells were infected directly with HIV-1 NL4.3 using 656, 1312, 2624, or 5248 TCID₅₀s per 1×10⁴ cells.

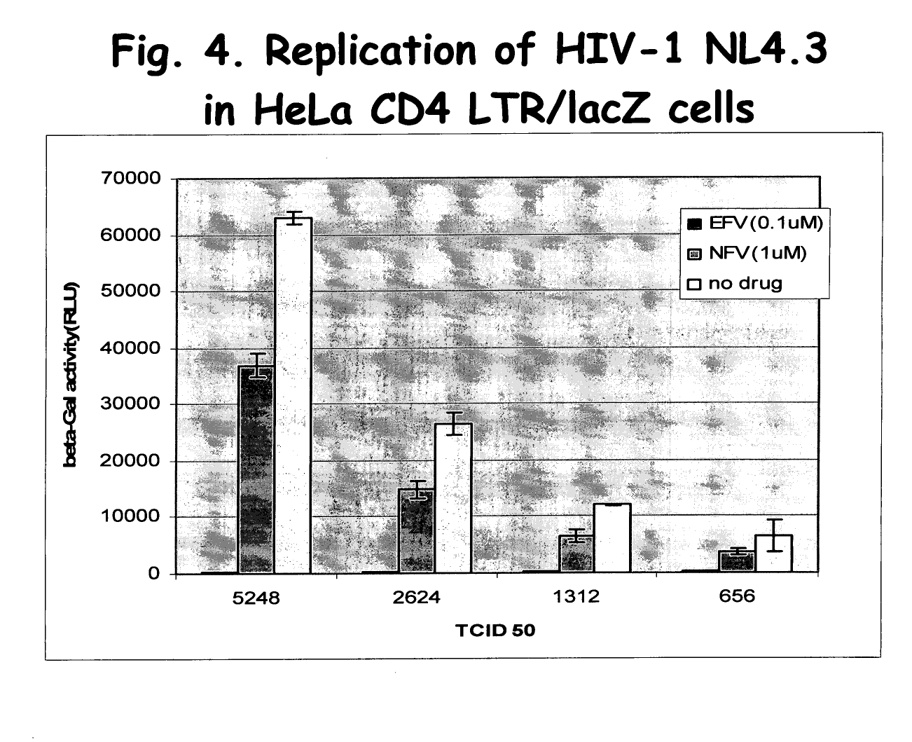

As with the co-culture assay, a significant induction of β-Gal activity was observed after directly infecting the HeLa CD4 LTR/lacZ indicator cells, which was dependent on viral TCID₅₀. At the highest virus TCID₅₀ (5248), a 247-fold induction of β-Gal activity was observed, with a maximum reporter gene signal of ˜63,000 RLUs measured (FIG. 4).

In these direct infection assays, >99% of the reporter signal was inhibited by >10×EC₉₀ concentrations of the non-nucleoside reverse transcriptase inhibitor EFV (FIG. 4). In contrast, this direct infection assay yielded only 42% inhibition of the reporter signal by 10×EC₉₀ concentrations of the protease inhibitor NFV (FIG. 4).

A significant fraction of the signal observed after direct infection of HeLa CD4 LTR/lacZ indicator cells with HIV-1 (58%) resulted from the initial round of infection. These results demonstrated that the known HeLa CD4 LTR/lacZ reporter cell assays possess limited sensitivity to late stage inhibitors of HIV-1 replication.

Examples 1-3 demonstrated that the present HIV co-culture assay has unexpected advantages over a representative, known HIV reporter cell assay. The reporter gene signal in the MT-2 and PM1 cell-based HIV co-culture assays was fully sensitive to late stage HIV-1 inhibitors. This was demonstrated by ≧98% inhibition in the presence of the protease inhibitor NFV.

These results indicate that the technical advantage of the present HIV co-culture assay extends far beyond that of known reporter cell assays. The increased sensitivity of the HIV co-culture assay to late stage inhibitors provides a method for the identification of a greater variety of such inhibitors in high throughput screens.

In addition, significantly higher reporter signals were observed in the MT-2 cell-based HIV co-culture assay format when compared the known HIV reporter cell assay. Up to 96-fold higher reporter gene signals were observed in the MT-2 cell-based HIV co-culture assay after infection with the lowest TCID₅₀ (656) when compared to direct infection of the HeLa CD4 LTR/lacZ indicator cells with the same TCID₅₀ (˜520,000 RLUs versus ˜6500 RLUs) (FIGS. 2 & 4).

### Example 4

### Susceptibility Assays Utilizing the MT-2 Cell-Based Co-culture Format

The HIV co-culture assay using MT-2 cells accurately and effectively evaluated the antiviral activities of HIV-1 inhibitors in susceptibility assays.

Half-log dilutions of test compounds were added to HeLa CD4 LTR/lacZ indicator cells seeded in 96-well plates at a cell density of 1×10⁴ cells per well in DMEM or RPMI (Life Technologies) containing 10% FBS (HyClone). MT-2 cells were infected with HIV-1 NL4.3 virus at an MOI of 0.08. Two hours after infection, infected cells were washed with RPMI, resuspended in RPMI medium, and added to the 96-well plates containing compound-treated or compound free HeLa CD4 LTR/lacZ indicator cells. Virus replication was measured 3 or 4 days after infection by quantifying HIV-1 Tat induced β-Gal activity in the HeLa CD4 LTR/lacZ indicator cells using the Dual-Light™ System according to the manufacturer's protocol (Applied Biosystems).

The antiviral activities of non-nucleoside reverse transcriptase inhibitors (CPV, DLV, EFV, and NVP), nucleoside analog reverse transcriptase inhibitors (AZT, ddl, 3TC, ADV, and d4T), integrase inhibitors (L-870810 and S-1360), protease inhibitors (NFV, SQV, and IDV), and an entry inhibitor (ATA) were evaluated in this MT-2 cell-based co-culture assay. Data was expressed as the percent of reporter gene activity in infected compound-treated cells relative to that of infected, compound-free cells. The 50% effective concentration (EC₅₀) was calculated as the concentration of compound that effected a decrease in the percentage of the virally encoded reporter gene activity in infected, compound-treated cells to 50% of that produced in infected, compound-free cells. In addition, the 90% effective concentration (EC₉₀) was calculated as the concentration of compound that effected a decrease in the percentage of the virally encoded reporter gene activity in infected, compound-treated cells to 90% of that produced in infected, compound-free cells.

EC₅₀ and EC₉₀ values were effectively and accurately measured with the MT-2 cell-based co-culture assay (Table 1). In addition, reference value ranges of EC₅₀s for these known inhibitors were obtained from the NIH anti-HIV therapeutics database (http://www.niaid.nih.gov/daids/dtpdb/). These reference ranges are included in Table 1 for comparison. The EC₅₀ values for the majority of the HIV inhibitors evaluated in the MT-2 cell-based co-culture assay were within the ranges reported in the literature.

TABLE 1
Antiviral activity of HIV-1 inhibitors in the MT-2 co-culture
assay.

| Inhibitor | | MT-2 Co-Culture Assay^{a} | | Literature^{b} |
|---|---|---|---|---|
| Class | Compound | EC₅₀ (μM) | EC₉₀ (μM) | EC₅₀ (μM) |
| NNRTI | EFV | 0.0014 | 0.0043 | 0.0005-0.006 |
| NNRTI | CPV | 0.0024 | 0.012 | 0.00069-0.0069 |
| NNRTI | NVP | 0.046 | 0.38 | 0.024-0.048 |
| NNRTI | DLV | 0.034 | 0.11 | 0.0001-0.01 |
| INTI | L-870810 | 0.0014 | 0.0059 | 0.004-0.008 |
| INTI | S-1360 | 0.088 | 1.09 | 0.11-2.6 |
| PI | NFV | 0.018 | 0.1 | 0.01-0.043 |
| PI | SQV | 0.012 | 0.026 | 0.0002-0.057 |
| PI | IDV | 0.026 | 0.055 | 0.0011-0.06 |
| NRTI | ADV | 0.039 | 0.22 | 0.04-0.5 |
| NRTI | AZT | 0.031 | 0.28 | 0.006-0.09 |
| NRTI | 3TC | 0.42 | 5.5 | 0.002-3.2 |
| NRTI | ddl | 1.7 | 14 | 0.46-10 |
| NRTI | d4T | 0.21 | 3.6 | 0.01-8 |
| Entry | ATA | 3.3 | 9.90 | 0.69-5 |
| Inhibitor | | | | |

^{a}Antiviral activity determined by measuring induced β-Gal activity 4 days after HIV-1 NL4.3 infection in the MT-2 cell-based co-culture assay.
^{b}Range of EC₅₀ values reported in the literature. Source: NIH anti-HIV therapeutics database.

These data demonstrate that the MT-2 cell-based co-culture assay provides an unexpectedly useful assay for measuring antiviral activity (e.g., EC₅₀ and EC₉₀ values) of HIV-1 inhibitors.

### Example 5

### Susceptibility Assays Utilizing the PM1 Cell-Based Co-culture Format

The HIV co-culture assay using PM1 cells accurately and effectively evaluated the antiviral activities of HIV-1 inhibitors in susceptibility assays.

These experiments were conducted by a modification of the method employed in Example 4. The modifications were as follows: PM1 cells were used in place of the MT-2 cells. The data was evaluated as described in Example 4.

The antiviral activities of non-nucleoside reverse transcriptase inhibitors (DLV, EFV, and NVP), nucleoside analog reverse transcriptase inhibitors (AZT, 3TC, and ADV), integrase inhibitors (L-870810 and S-1360), and protease inhibitors (NFV, SQV, and IDV), were evaluated in the PM1 cell-based co-culture assay. The results are shown in Table 2.

In addition, reference value ranges of EC₅₀s for these known inhibitors were obtained from the NIH anti-HIV therapeutics database (http://www.niaid.nih.gov/daids/dtpdb/). These reference ranges are included in Table 2 for comparison. The EC₅₀ values for the majority of the HIV inhibitors evaluated in the MT-2 cell-based co-culture assay were within the ranges reported in the literature.

TABLE 2
Antiviral activity of HIV-1 inhibitors in the PM-1 co-culture
assay.

| Inhibitor | | PM-1 Co-Culture Assay^{a} | | Literature^{b} |
|---|---|---|---|---|
| Class | Compound | EC₅₀ (μM) | EC₉₀ (μM) | EC₅₀ (μM) |
| NNRTI | EFV | 0.00019 | 0.0062 | 0.0005-0.006 |
| NNRTI | DLV | 0.0059 | 0.10 | 0.0001-0.01 |
| NNRTI | NVP | 0.012 | 0.26 | 0.024-0.048 |
| INTI | L-870810 | 0.00061 | 0.0024 | 0.004-0.008 |
| INTI | S-1360 | 0.091 | 0.28 | 0.11-2.6 |
| PI | NFV | 0.010 | 0.058 | 0.01-0.043 |
| PI | SQV | 0.0053 | 0.034 | 0.0002-0.057 |
| PI | IDV | 0.012 | 0.047 | 0.0011-0.06 |
| NRTI | ADV | 0.025 | 0.15 | 0.04-0.5 |
| NRTI | AZT | 0.030 | 0.18 | 0.006-0.09 |
| NRTI | 3TC | 0.18 | 1.7 | 0.002-3.2 |

^{a}Antiviral activity determined by measuring induced β-Gal activity 4 days after HIV-1 NL4.3 infection in the PM-1 cell-based co-culture assay.
^{b}Range of EC₅₀ values reported in the literature. Source: NIH anti-HIV therapeutics database.

These data demonstrate that the PM1 cell-based co-culture assay provides an unexpectedly useful assay for measuring antiviral activity (e.g., EC₅₀ and EC₉₀ values) of HIV-1 inhibitors.

Additional Conclusions From Examples 4 and 5

The results of the experiments reported in Examples 4 and 5 demonstrate utility of the present HIV co-culture assays that extends significantly beyond that of known HIV reporter cell and reporter virus assays. The observation of nearly complete inhibition (≧98%) of reporter signal in the HIV co-culture assay by late stage inhibitors (e.g., protease inhibitors) allows for the accurate determination of EC₉₀ values for such inhibitors in susceptibility assays (Tables 1 & 2). This contrasts with the most commonly used commercially available HIV reporter cell (Antivirogram™) or reporter virus assays (PhenoSense™), which are not commonly used for determining EC₉₀ values.

### Example 6

### High Levels of Replication of HIV-1 in MT-2 and PM1 T-Cell Lines

The present HIV co-culture assay employs Vif-nonpermissive target cells, which makes it suitable for detecting inhibitors of the function of the HIV protein Vif.

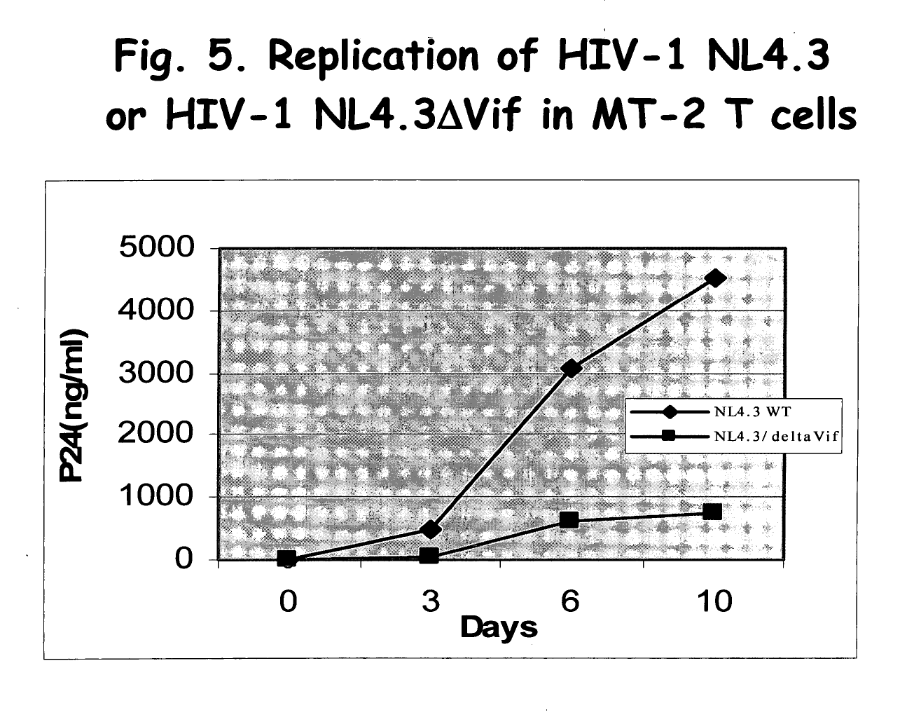

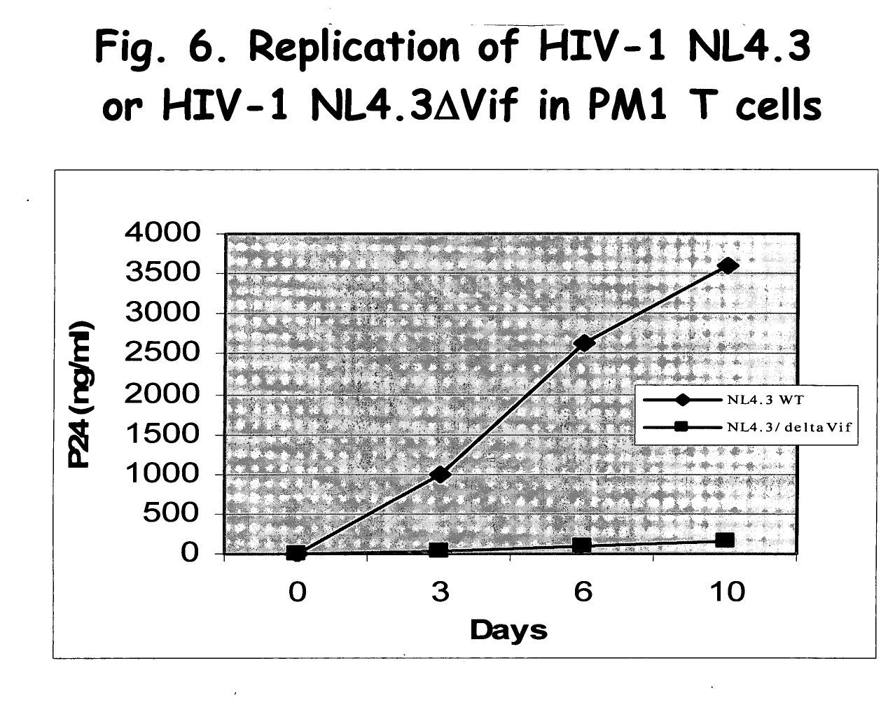

The replication kinetics of wild-type HIV-1 NL4.3 (NL4.3 wt) was compared to that of an HIV-1 NL4.3 mutant virus which contains a deletion in Vif coding sequences (NL4.3/ΔVif) in MT-2 and PM1 cells. MT-2 cells or PM1 cells were infected with NL4.3 virus or NL4.3/ΔVif virus using equivalent mois of 0.16 for 2 hours. Infected cultures were then washed with RPMI resuspended in 5 ml of RPMI medium at final cell densities of 2×10⁵ cells/ml. The infected cells were incubated at 37° C. with 5% CO₂ and 1 ml of the cell-free culture supernatants were collected immediately after infection (day 0) or 3, 6 or 10 days post infection. Virus replication was measured by quantifying HIV-1 p24 antigen present in the supernatants of infected cell cultures using the COULTER™ HIV-1 p24 antigen assay kit (Beckman Coulter, Miami, Fla.) according to the manufacturer's protocol. Data were plotted as ng/ml of p24 antigen detected versus days post infection (FIGS. 5 and 6).

Suitable cells for this type of assay were identified by comparing the replication kinetics of wild-type (wt) HIV-1 NL4.3 to that of an HIV-1 NL4.3 mutant virus which contained a deletion in Vif coding sequences (HIV-1 NL4.3/ΔVif). FIGS. 5 & 6 illustrate levels of virus replication achieved in MT-2 or PM1 cells infected with HIV-1 NL4.3 wt or HIV-1 NL4.3/ΔVif using equivalent multiplicities of infection (moi=0.16). Virus replication was measured by quantifying p24 production in the supernatants of infected cells either immediately after infection (day 0) or 3, 6, or 10 days after infection using an HIV-1 p24 antigen assay kit (Beckman Coulter).

As shown in FIGS. 5 & 6, significantly higher levels of p24 were produced in the supernatants of MT-2 or PM1 cells infected by HIV-1 NL4.3 wt when compared to the same cells infected with HIV-1 NL4.3/ΔVif. In MT-2 cells, 6-fold higher levels of p24 were measured in cells infected with HIV-1 NL4.3 wt when compared to the same cells infected with HIV-1 NL4.3/ΔVif 10 days after infection. In PM1 cells, 21-fold higher levels of p24 were measured in cells infected with HIV-1 NL4.3 wt when compared to the same cells infected with HIV-1 NL4.3/ΔVif 10 days after infection.

These data demonstrate that both MT-2 cells and PM1 cells exhibit a Vif-nonpermissive phenotype. Thus, these cells can provide an HIV co-culture assay that includes HIV-1 Vif as an antiviral target.

One advantage of the HIV co-culture assay format is target cell flexibility. The preferred target cells for the HIV co-culture assay could be selected to include the maximum number of novel HIV-1 antiviral targets in the assay. Therefore, the HIV co-culture assay was designed to include Vif as a target by utilizing target cells that exhibited a Vif-nonpermissive phenotype.

### Example 7

### MT-2 and PM1 Cell-Based HIV-1 Co-Culture Assays Detect Inhibition of Activity of HIV-1 Vif

HIV-1 replication in the MT-2 or PM1 cell-based co-culture assay was shown to be dependent on a functional Vif gene.

These experiments were conducted by a modification of the methods employed in Examples 1-3. Replication of wild-type (wt) HIV-1 NL4.3 was compared to that of an HIV-1 NL4.3 mutant virus, which contains a deletion in Vif coding sequences (HIV-1 NL4.3/ΔVif), in the MT-2 and PM1 cell-based co-culture assays. MT-2 or PM1 T-cells were infected with HIV-1 NL4.3 or HIV-1 NL4.3/ΔVif using equivalent multiplicities of infection (moi=0.08). Two hours after infection, infected MT-2 or PM1 cells were washed with RPMI and added to the 96-well microtiter plates containing the HeLa CD4 LTR/lacZ indicator cells (seeded at 1×10⁴ cells/well) at final infected MT-2 or PM1 cell densities of 2×10⁴ cells/well. Alternatively, HeLa CD4 LTR/lacZ indicator cells (seeded at 1×10⁴ cells/well) were infected directly with HIV-1 NL4.3 or HIV-1 NL4.3/ΔVif using equivalent mois (moi=0.08). Virus replication was measured 4 days after infection by quantifying HIV-1 Tat induced β-Gal activity in the HeLa CD4 LTR/lacZ indicator cells using the Dual-Light™ System according to the manufacturer's protocol (Applied Biosystems). Data were expressed as relative light units (RLUs), which correspond to counts per second measured using a Perkin Elmer Victor 2 luminometer. All experiments were performed with 3 or more replicates.

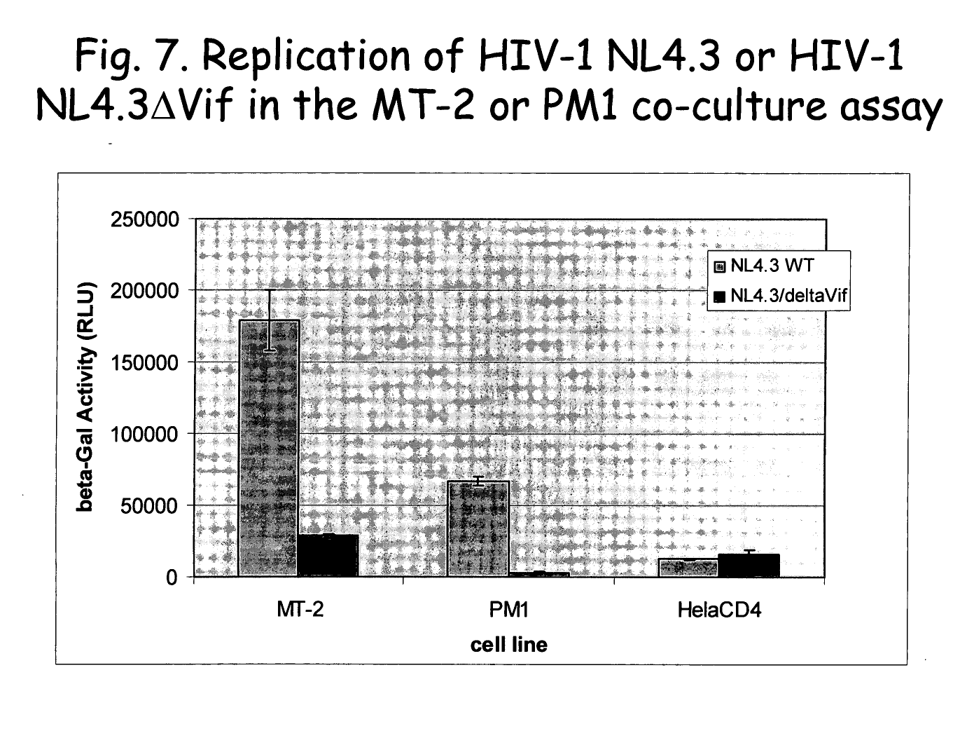

As shown in FIG. 7, wt NL4.3 virus replicated more efficiently when compared to the NL4.3/ΔVif mutant virus in the MT-2 and PM1 cell-based co-culture assays. In the MT-2 cell-based co-culture assay, a 78% reduction in virus replication was observed for the NL4.3/ΔVif mutant virus when compared to wt, while a 95% reduction in NL4.3/ΔVif mutant virus replication was observed in the PM1 cell-based co-culture assays. As expected, nearly identical levels of virus replication were measured after direct infection of the HeLa CD4 LTR/lacZ cells (Vif permissive cells) with wt NL4.3 or the NL4.3/ΔVif mutant virus. These results confirm that maximum levels of virus replication in the MT-2 or PM1 cell-based HIV-1 co-culture assays depend on a functional Vif gene.

Therefore, the MT-2 and PM1 cell-based co-culture assays represent novel high throughput assays useful for screening inhibitors of HIV-1 Vif function.

### Example 8

### A High Throughput Screening System Employing the MT-2 Cell-Based HIV-1 Co-Culture Assay

The HIV co-culture assay was used to identify inhibitors in a high throughput screen format. Thirteen 96-well trial screening plates were evaluated in the MT-2 cell-based co-culture assay. The trial plates were designed to mimic an actual compound library screen and thus, contained DMSO in each well at a final concentration equivalent to that encountered in a typical cell-based screen (1% final).

HeLa CD4 LTR/lacZ indicator cells were seeded in thirteen 96-well plates at a cell density of 1×10⁴ cells per well in DMEM (Life Technologies) containing 10% FBS (HyClone). DMSO (Sigma-Aldrich, St. Louis, Mo.) was then added to at a final concentration of 1%.

In addition to DMSO, test compound was added to 11 of the trail screening plates in 6-12 wells per plate at final concentrations corresponding to EC₅₀, EC₉₀ or 2×EC₉₀ compound concentrations. Compounds in this trial screen include: ADV (NRTI), L-870810 (INI), EFV (NNRTI), CPV (NNRTI) or NFV (PI) introduced at their respective ECSO or EC₉₀ concentrations or L-870810 (INI), CPV (NNRTI) and NFV (PI) introduced at their respective 2×EC₉₀ concentrations. EC₅₀, EC₉₀, or 2×EC₉₀ values for each compound were determined based on experiments described in Table 1. All 96-well trial screening plates were formatted such that 3 wells represented the no drug control wells, 3 wells represented the no virus control wells, and 90 wells represented test wells.

MT-2 cells were infected with HIV-1 NL4.3 virus using an moi of 0.02. Two hours after infection, infected cells were washed with RPMI and resuspended in RPMI medium and then added to the 96-well plates containing compound-treated or compound free HeLa CD4 LTR/lacZ indicator cells. Virus replication was measured 4 days after infection by quantifying HIV-1 Tat induced β-Gal activity in the HeLa CD4 LTR/lacZ indicator cells using the Dual-Light™ or Galcto-Star™ reporter gene assay system according to the manufacturer's protocol (Applied Biosystems). Antiviral maximum signals (AV Max) and antiviral minimum signals (AV Min) were calculated from the 2 plates containing DMSO only and represent the mean values measured from the 6 no drug control wells and the 6 no virus control wells, respectively.

The standard deviations (SD) across 90 wells on each of 2 plates containing DMSO only were calculated and divided by the AV Max to obtain the coefficient of variation values (CV). In addition, the Z′ coefficient was calculated from the no drug control wells using the equation 1-[(3×SD AV Max)−(3×SD AV Min)/(AV max−AV Min)] (Zhang et al., J. Biomolec. Screen. 4:67-73 (1999)). In the 11 trial screening plates containing test compounds, data from the reporter gene measurements were expressed as the percent inhibition of reporter gene activity in infected compound-treated cells relative to that of infected compound-free cells. Wells that exhibited ≧50% inhibition of the reporter gene signal relative to the no-compound control wells (AV max) were scored as hits. Data were then analyzed to determine the number of wells containing known inhibitors that scored as hits relative to the total number of inhibitors present.

As an initial demonstration of assay suitability for high throughput screening, signal-to-background values, coefficients of variation (CV), and screening window coefficients (Z′ value) were calculated for the MT-2 cell-based co-culture assay. The results showed antiviral maximum (AV Max) and antiviral minimum (AV Min) signals of 590,807 and 24,022 RLUs, respectively (Table 3). The AV Max value was divided by the AV Min value to yield a signal-to-background of 25 (Table 3).

TABLE 3
HIV co-culture screen parameters.

| Parameter | Value |
|---|---|
| AV Max^{a} | 590,807 |
| AV Min^{b} | 24,022 |
| AV Max/AV Min | 25 |
| CV^{c} | 19% |
| Z^{d} | 0.76 |

^{a}AV Max: mean of no drug control wells taken from 2 mock screen plates (n = 6). Value expressed in relative light units (RLUs).
^{b}AV Min: mean of no virus control wells taken from 2 mock screen plates (n = 6). Value expressed in relative light units (RLUs).
^{c}CV = 100 × (SD test wells/AV Max)
^{d}Z = 1 − [(3 × SDAV Max − 3 × SDAV Min)/AV Max − AV Min)]

As shown in Table 3, the MT-2 cell-based co-culture assay exhibited CV values of 19% across the 2 mock screening plates used for these calculations. In addition, Z′ values were determined for the assay. The Z′ value is reflective of the dynamic range as well as the variation of the assay and is a useful tool for assay comparisons and assay quality determinations (Zhang et al., supra). Z′ values were calculated using designated control wells and typically a Z′ value >0.5 is considered favorable for high throughput screening. As shown in Table 3, the MT-2 cell-based co-culture assay exhibited a Z′ value of 0.76.

Evaluating the 11 additional 96-well trial screening plates further demonstrated the robustness of the MT-2 cell-based co-culture assay in high throughput screening. The trail screening plates in this experiment contained DMSO in each well at a final concentration equivalent to that encountered in a typical cell-based screen (1% final). In addition, the trial plates contained known HIV-1 inhibitors of different classes distributed randomly in 6-12 wells per plate. Data representing the reporter gene measurements from trial screening plates were analyzed as the percent inhibition of reporter gene activity in infected compound-treated wells relative to that of infected compound-free wells. Wells exhibiting ≧50% inhibition of the reporter gene activity compared to no compound control wells were scored as hits.

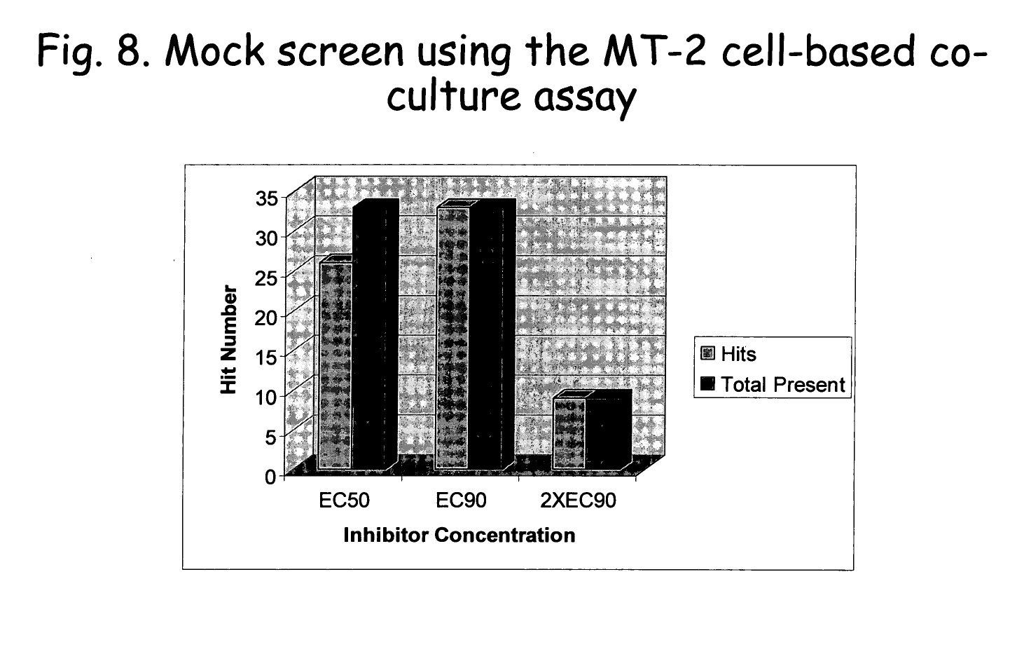

Hits identified in the trial screen were then plotted relative to the number of total inhibitors present (FIG. 8). The results showed that 79% of the inhibitors present at their respective EC₅₀ concentrations were identified as antiviral hits in the MT-2 cell-based trail screen. In addition, 100% of the compounds present at their respective EC₉₀ or 2×EC₉₀ concentrations were identified as hits in the MT-2 cell-based mock screen (FIG. 8).

The significant signal-to-background ratio combined with a CV <20% and a favorable Z′ value confirm that the MT-2 cell-based co-culture assay is suitable for high throughput screening. The data demonstrate that the MT-2 cell-based co-culture assay reproducibly identified diverse classes of HIV-1 inhibitors when present in concentration ranges that are expected to inhibit HIV-1 replication.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

All publications and patent applications in this specification are indicative of the level of ordinary skill in the art to which this invention pertains.

The invention has been described with reference to various specific and preferred embodiments and techniques. However, it should be understood that many variations and modifications may be made while remaining within the spirit and scope of the invention.

1. A co-culture method for detecting replication of a lentivirus, comprising:
infecting Vif-nonpermissive cells with the lentivirus in vitro;
co-culturing the infected Vif-nonpermissive cells and indicator cells;
contacting the co-culture with a test compound; and
assaying for the indicator cells in the co-culture.

2. The method of claim 1, wherein the infecting step comprises infecting a Vif-nonpermissive cell selected from the group consisting of PM-1, MT-2, H9, Hut78, 174XCEM, C8166, MT-4, or Jurkat cells.

3. The method of claim 1, wherein contacting the co-culture with a test compound comprises adding the test compound to the Vif-nonpermissive cells before co-culturing.

4. The method of claim 1, wherein assaying for the indicator comprises adding an enzyme substrate to the co-culture.

5. The method of claim 1, wherein assaying for the indicator comprises assaying for chemiluminescence.

6. The method of claim 1, wherein assaying for the indicator comprises lysing cells of the co-culture.

7. The method of claim 1, wherein the lentivirus comprises HIV.

8. The method of claim 1, wherein the lentivirus comprises SIV, Caprine arthritis encephalitis virus, bovine immunodeficiency virus, or Visna/maedi virus.

9. The method of claim 1, wherein the test compound comprises an HIV-1 inhibitor.

10. A method for detecting an inhibitor of activity of lentivirus Vif, comprising:
infecting Vif-nonpermissive cells with the lentivirus in vitro;
co-culturing the infected Vif-nonpermissive cells and indicator cells;
contacting the co-culture with a test compound;
assaying for the indicator in the co-culture; wherein indicator below a threshold level indicates inhibition of lentivirus replication by the test compound and that the test compound is a lentivirus replication inhibitor; and
challenging Vif independent lentivirus replication assay with the lentivirus replication inhibitor; lentivirus replication above second threshold level indicating inhibition of Vif-activity by the lentivirus replication inhibitor and that the lentivirus replication inhibitor is the inhibitor of activity of lentivirus Vif.

11. The method of claim 10 wherein challenging the Vif independent lentivirus replication assay with the lentivirus replication inhibitor comprises assaying the lentivirus replication inhibitor against the lentivirus in a Vif-permissive cell.

12. The method of claim 10, wherein challenging the Vif independent lentivirus replication assay with the lentivirus replication inhibitor comprises assaying the lentivirus replication inhibitor against a replication competent lentivirus lacking a functional Vif-gene.

13. The method of claim 10, wherein the replication competent lentivirus lacking a functional Vif-gene comprises: lentivirus analogous to lentivirus that includes a functional Vif-gene; lentivirus that is different strain of lentivirus that includes functional Vif-gene; lentivirus related to lentivirus that includes a functional Vif-gene; or combination thereof.

14. A method for indicating replication of HIV, comprising:
adding HeLa CD4 LTR/lacZ indicator cells to a vessel;
adding test compound to the vessel;
contacting MT-2 or PM1 Vif-nonpermissive cells with HIV;
incubating the Vif-nonpermissive cells and HIV for about 1 to about 4 hours;
washing the incubated Vif-nonpermissive cells with cell culture medium;
adding the washed Vif-nonpermissive cells to the vessel containing the indicator cells to form a mixture of Vif-nonpermissive cells, indicator cells, and test compound;
co-culturing the mixture for about 1 to about 8 days; and
monitoring the culture for β-galactosidase activity; wherein the level of β-galactosidase activity indicates the level of HIV in the culture.

15. A method for detecting an inhibitor of activity of HIV Vif, comprising:
adding HeLa CD4 LTR/lacZ indicator cells to a vessel;
adding test compound to the vessel;
contacting MT-2 or PM1 Vif-nonpermissive cells with HIV;
incubating the Vif-nonpermissive cells and HIV for about 1 to about 4 hours;
washing the incubated Vif-nonpermissive cells with cell culture medium;
adding the washed Vif-nonpermissive cells to the vessel containing the indicator cells to form a mixture of Vif-nonpermissive cells, indicator cells and test compound;
co-culturing the mixture for about 1 to about 8 days;
monitoring the culture for β-galactosidase activity; wherein activity below a threshold level indicates inhibition of HIV replication by the test compound and that the test compound is lentivirus replication inhibitor; and
challenging Vif independent lentivirus replication assay with the lentivirus replication inhibitor;
lentivirus replication above second threshold level indicating inhibition of Vif-activity by the lentivirus replication inhibitor and that the lentivirus replication inhibitor is the inhibitor of activity of lentivirus Vif.
